(19) United States
(12) Patent Application Publication
(10) Pub. No.: US 2023/0160883 A1
(43) Pub. Date: May 25, 2023

(54) Tomato Zonate Spot Virus Detection Reagent

(71) Applicant: Yunnan Academy of Tobacco Agricultural Sciences, Kunming City (CN)

(72) Inventors: Ning JIANG, Kunming City (CN); Zhenyuan XIA, Kunming City (CN); Xiaotong GAI, Kunming City (CN); Zhonglong LIN, Kunming City (CN); Canhua LU, Kunming City (CN); Changjun HUANG, Kunming (CN); Junhong MA, Kunming City (CN); Xiaohan MO, Kunming City (CN); Wei WU, Kunming City (CN); Chunming LIU, Kunming City (CN); Qiuqiang HOU, Kunming City (CN); Jian GUO, Kunming City (CN)

(21) Appl. No.: 17/947,411

(22) Filed: Sep. 19, 2022

(30) Foreign Application Priority Data
Nov. 19, 2021 (CN) 202111399298.4

(51) Int. Cl. G01N 33/531; C12N 5/12; G01N 33/543; G01N 33/569

(52) U.S. Cl. CPC G01N 33/531; C12N 5/12; G01N 33/54388; G01N 33/56983; C12N 2510/02; G01N 2333/08

(57) ABSTRACT

This invention provides a tomato zonate spot virus detection reagent. Optimized gene encoding N protein of TZSV is expressed and purified to obtain N protein of TZSV, and two specific hybridoma cell lines are obtained, from which specific anti-TZSV monoclonal antibody could be obtained, respectively. Test strip for detecting tomato zonate spot virus is prepared with the monoclonal antibody. It is proven by experiments that the test strip of this invention is specific for TZSV, and can realize rapid detection of TZSV in leaves and other samples, with high sensitivity and convenient operation.

### CROSS-REFERENCE TO RELATED APPLICATION

This application claims priority to and the benefit of Chinese Application No. 202111399298.4 filed Nov. 19, 2021. The entire disclosure of Chinese Application No. 202111399298.4 is incorporated herein by reference.

### TECHNICAL FIELD

The present invention relates to the technical field of plant virus detection, in particular to a Tomato zonate spot virus (TZSV) detection reagent.

### BACKGROUND ART

Tomato zonate spot virus (TZSV) belongs to Orthotospovirus genus, it was first discovered in China in 2005 and is mainly distributed in Yunnan and Guangxi provinces and other regions. It affects tobacco, pepper, tomato and other crops. The virus can cause spotted wilt disease, and even fail to harvest in serious cases, resulting in significant economic losses to agricultural production. Jin Yan Tian et al. prepared a monoclonal antibody to TZSV N protein and a test strip. However, there is still no commercial test strip for TZSV in market.

### SUMMARY OF INVENTION

Purpose of the present invention is to provide a colloidal gold detection reagent for tomato zonate spot virus (TZSV).

In order to achieve the purpose of the invention, Optimized gene encoding the N protein of TZSV virus (SEQ ID NO:2) was constructed into Escherichia coli expression system, and N protein of TZSV virus was obtained by expression and purification; then, 6-week-old female BALB/c mice were immunized with the TZSV N protein, and splenocytes of the immunized mice were fused with myeloma cells SP2/0.

Two specific hybridoma cell lines were obtained by screening, whose depository numbers are CCTCC No. C202160 and CCTCC No. C202196, respectively.

Specific anti-TZSV monoclonal antibody could be obtained from above two hybridoma cell lines.

Anti-TZSV monoclonal antibody could be obtained as follows: resuscitating hybridoma cells, preparing ascites, and purifying ascites to obtain specific monoclonal antibody.

The monoclonal antibody obtained by the invention can be used to prepare tomato zonate spot virus detection reagent.

In an example of the invention, a tomato zonate spot virus (TZSV) colloidal gold test strip (TZSV hand-held colloidal gold rapid test strip) is provided. the test strip consists of a sample pad, a gold labeled pad, a nitrocellulose membrane, an absorbent pad and a backplane; sample pad, gold labeled pad, nitrocellulose membrane and absorbent pad (absorption pad) are successively superimposed and pasted on the backplane, and each part is superimposed between each other by 2-3 mm; the gold labeled pad was coated with colloidal gold labeled anti-TZSV monoclonal antibody; nitrocellulose membrane is provided with a test line and a quality control line (T line and C line), the test line coated with anti-TZSV monoclonal antibody, the quality control line coated with anti-mouse IgG secondary antibody; Wherein, the anti-TZSV monoclonal antibody (murine anti-TZSV monoclonal antibody) is generated by hybridoma cell lines with depository numbers of CCTCC No. C202160 and CCTCC No. C202196.

The hybridoma cell lines of anti-TZSV monoclonal antibody were obtained as follows: gene encoding N protein of TZSV virus (SEQ ID NO:2) was optimized and constructed into Escherichia coli expression system, and the recombinant TZSV virus was obtained by expression and purification.; the 6-week-old female BALB/c mice were immunized with the recombinant TZSV virus, and then splenocytes of the immunized mice were fused with myeloma cells SP2/0 to obtain specific monoclonal antibody cell lines.

Preferably, the sample pad is a glass cellulose film.

Preferably, concentration of colloidal gold labeled anti-TZSV monoclonal antibody coated on the gold labeled pad is 0.3-0.5 mg/ml.

Preferably, concentration of anti-TZSV monoclonal antibody coated on the test line is 0.5-1.5 mg/ml.

Preferably, concentration of anti-mouse IgG secondary antibody coated on the quality control line is 1 mg/ml.

Preferably, size of the sample pad is 3 mm × 15 mm, size of the gold labeled pad is 3 mm × 3 mm, size of the nitrocellulose membrane is 3 mm × 28 mm, and size of the absorbent pad is 3 mm × 19 mm.

Preferably, space between the test line and the quality control line is 6-6.5 mm.

Preferably, the backplane (bottom plate) is a PVC board.

Structure diagram of the TZSV virus hand-held colloidal gold rapid test strip of the invention is shown in FIG. 1. wherein, 1 is result observation area, 2 is marking area, 3 is quality control area (C), 4 is test area (T), 5 is sample adding area.

Secondly, the invention provides use of the test strip in detecting TZSV virus.

Specific detection procedure and principle of the TZSV handheld colloidal gold rapid test strip of the invention are as follows:

first, put the rapid test strip vertically into test sample, not exceeding position of arrow (FIG. 1); the sample solution permeates the test strip with colloidal gold labeled antibody along the sample pad, if the sample solution contains TZSV, the TZSV will bind to the anti-TZSV virus monoclonal antibody on the gold labeled pad and mouse anti-TZSV monoclonal antibody on the NC film, after 8-10 min, color change of the test area can be observed in the observation area, i.e., positive band appears; if the sample solution does not contain TZSV, then no positive band can be observed in the test area. Regardless of whether TZSV was contained in the sample solution or not, when the sample solution reached the nitrocellulose membrane, the mouse anti-TZSV monoclonal antibody on the gold labeled test strip could bind to the anti-mouse IgG secondary antibody coated in the control area to produce color reaction.

Therefore, judgment standard of results of the TZSV handheld colloidal gold rapid test strip of the present invention is:
Negative results (-), only one C line appeared;
Positive result (+) : T line and C line appear simultaneously.

The TZSV virus hand-held colloidal gold rapid test strip of the invention has good anti-interference effect on leaf and other samples, and can be widely used in rapid detection of TZSV, with the following unique advantages:
(1) Simple operation, no special instruments and equipment, it is easy to directly test in field and other sites.
(2) Short detection time, 3-5 minutes to complete the detection; determination criteria of results was uniform, that is, negative results (-), only one C line appears; Positive (+) : T line and C line appear simultaneously.
(3) High detection sensitivity.

### DESCRIPTION OF DRAWINGS

FIG. 1 is structure diagram of the TZSV handheld colloidal gold rapid test strip of the present invention; wherein, 1 is the result observation area, 2 is the marking area, 3 is the quality control area (C), 4 is the test area (T), 5 is the sample adding area.

FIG. 2 shows specific detection of the TZSV test strip of the present invention. From left to right, leaf samples positive for tobacco viruses TMV, PVY, TSWV, ChiVMV and TZSV determined by RT-PCR are shown.

FIG. 3 shows electrophoretic diagram of PET28A-TZSV-N protein purification in example 6 of the present invention.

FIG. 4 shows the 9# monoclonal antibody standard curve of example 6 of the present invention.

FIG. 5 shows sensitivity test result of TZSV test strip in example 6 of the present invention.

FIG. 6 shows specific detection result of the TZSV test strip of example 6 of the present invention.

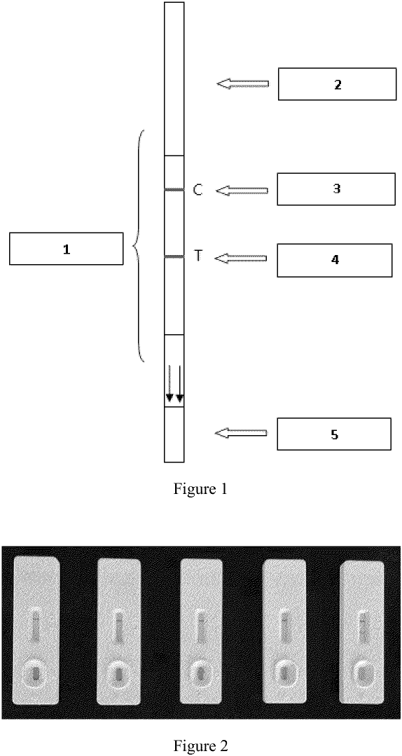

### EMBODIMENTS

The following examples are used to illustrate the invention without limiting scope of the invention. Unless otherwise specified, technical means used in the examples are conventional means well known to a person skilled in the art, and the raw materials used are commercially available goods.

### Example 1. Expression and Purification of TZSV Protein

### Test Expression of Protein

1. Sequence synthesis: According to protein sequence, optimized sequence was synthesized and cloned into vector pET30a, and the sequence was synthesized by Sangon Bioengineering (Shanghai) Co., LTD. Amino acid sequence of tomato zonate spot virus protein was shown as SEQ ID NO: 1. Nucleic acid sequence of the optimized tomato zonate spot virus gene is shown as SEQ ID NO:2.

2. Strain activation: PET30a-TZSV plasmid was transformed into BL21 (DE3) and coated on LB solid medium (Kanamycin concentration of 50 µg/mL). Next day, monoclonal colonies were selected and added into 5 mL LB liquid medium (Kanamycin concentration 50 µg/mL) and cultured at 37° C. for 12 h-14 h. Original sequence of protein was SEQ ID NO: 1.

3. Small test expression: next day, strains were added into 5 mL LB liquid medium at 1:50 (Kanamycin concentration of 50 µg/mL) and cultured at 37° C. until OD=0.4-0.6. After centrifugation, 1 mL of bacterial solution was absorbed and centrifuged for using as control before induction. 4 mL of bacterial solution was added with 0.8 mM IPTG, induced and expressed at 25° C. for 6 h, after that, the bacterial solution was centrifuged at 8000 RPM and 4° C. for 1 min and collected.

4. Identification of protein expression form: above expressed bacteria were added to 1 mL crushing solution for ultrasonic lysis. Lysis conditions: temperature ice bath, power 40%, ultrasonic 2 s, 2 s interval, time 30 min. Supernatant and precipitate were collected after centrifugation at 12000 rpm and 4° C. for 1 min. Result of SDS-PAGE showed that target protein was mainly expressed in soluble form.

### Mass Expression and Purification of Protein

1. Strain activation: pET30a-TZSV colonies were selected from solid plates and added into 5 mL LB liquid medium (Kanamycin concentration 50 µg/mL), and incubated at 37° C. for 12 h-14 h.

2. Small test expression: next day, strains were added into 800 mL LB liquid medium at 1:50 (Kanamycin concentration of 50 µg/mL), cultured at 37° C. to OD=0.4-0.6, and IPTG was added at 0.8 mM. After induction and expression at 25° C. for 6 h, bacteria was centrifuged at 8000 RPM and 4° C. for 15 min and collected.

3. Strain lysis: 100 mL crushing solution was added for ultrasonic lysis. Lysis conditions: temperature ice bath, power 60%, ultrasonic 2 s, interval 2 s, time 15 min. Supernatant and precipitate were collected after centrifugation at 12000 RPM and 4° C. for 15 min.

4. Purification of supernatant: The collected supernatant was purified by high affinity Ni resin, and the flowing through fluid and eluent were collected. Results of SDS-PAGE showed that 50 mM imidazole elution had best purity of protein. The imidazole was removed by dialysis with 50 mM imidazole eluent, and effect of dialysis was detected by SDS-PAGE, and results showed that purity and concentration of protein after dialysis were feasible. After detection, finally, purity of target protein was more than 90%, concentration was 2 mg/mL, and protein amount was 10 mg.

### Results

Expression conditions of pet30a-tzsv protein were as follows: IPTG concentration was 0.8 mM, induction temperature is 25° C., induction time is 6 h. Protein was mainly expressed in supernatant, which was purified by Ni column and dialyzed to remove imidazole, and in final, TZSV N protein was obtained with concentration of 2 mg/mL, purity more than 90%, protein of 10 mg.

### Example 2. Antibody Preparation

### Preparation of Monoclonal Antibody

1. Immunogen preparation: The expressed and purified protein was mixed and emulsified with equal volume of Freund’s adjuvant to form a water-in-oil state for immunizing mice.

2. Immunization strategy: 4 Balb/c mice were immunized with protein for 3 times subcutaneously, with an interval of 4 weeks, and finally titer was detected by indirect ELISA.
The steps of indirect ELISA method are as follows:
   1) The purified protein was diluted with 0.1 mol/L carbonate buffer, pH=9.6 to 1 µg/ml, then added to 96-well microplate, 100 µl in each well, and reacted at 37° C. for 3 h or placed at 4° C. overnight.
   2) Liquid in the plate well was shaken off, and 250 µl washing buffer was added to stand for 30 s, then liquid in the plate was shaken off. Such operation was repeated for 3 times.
   3) test samples were added with 100 µl to each well, and positive control (positive mouse serum obtained in step (2)), negative control (mouse serum before immunization) and blank control (without mouse serum) were added simultaneously for reacting at 37° C. for 45 min.
   4) Repeat step 2);
   5) HRP labeled sheep anti-mouse enzyme-conjugated secondary antibody was added with 100 µl per well, and react at 37° C. for 45 min.
   6) Repeat step 2);
   7) chromogenic agent was added with 100 µl to each well, and react at room temperature for 15 min in the dark.
   8) stop solution was added with 100 µl in each well, and OD value was read at wavelength 450 using a microplate reader. Antiserum titer is shown in Table 1:

TABLE 1

| | | | | | | | | | | |
|---|---|---|---|---|---|---|---|---|---|---|
| Animal number | 100 | 1K | 3K | 9K | 27K | 81K | 243K | Negative control | Titer | Immunization schedule |
| M210045 | 3.9064 | 3.7261 | 3.7004 | 3.6867 | 3.0881 | 2.472 | 1.7417 | 0.1373 | > 1:243000 | secondary immunization |
| M210046 | 3.3312 | 3.1428 | 3.0632 | 2.3105 | 1.5738 | 0.8598 | 0.4183 | 0.1461 | > 1:243000 | secondary immunization |
| M210047 | 3.1319 | 2.5112 | 1.6485 | 0.9701 | 0.5058 | 0.2833 | 0.1967 | 0.1372 | > 1:81000 | secondary immunization |
| M210048 | 3.4423 | 3.4091 | 3.126 | 2.7418 | 2.0721 | 1.193 | 0.5778 | 0.1252 | > 1:243000 | secondary immunization |

3. Cell fusion: two weeks after the last immunization, intraperitoneal injection of antigen was performed for booster immunization, and cell fusion was performed three days later. Mice were sacrificed by cutting their neck, soaked in 70% ethanol for 30 min for disinfection, and their abdominal cavity was cut open in the ultra-clean table. Spleen was removed, ground, and passed through 80 mesh screen to obtain splenocytes. SP2/0 myeloma cells were added, and cell fusion was carried out under action of PEG4000.

4. Fusion screening: The fused cells were spread into a 96-well plate and cultured with HAT medium. After three days, medium was changed to HT medium. After 10 days, cell culture supernatant was taken for detection.

5. Cloning and strain building: use the limited dilution method to clone the positive wells, 10 days later, the positive clones continue to be cloned by the limited dilution method, until the clones are all positive, positive cell lines can be established. Finally, 5 positive cell lines were obtained.

6. Expanded culture: monoclonal cells of the established strain were expanded cultured and stored by freezing.

### Preparation and Purification of Ascites

1. Preparation of ascites: one week in advance, mice were intraperitoneally injected with mineral oil, and a certain number of cells were injected into mice., about 10 days later, ascites was collected and centrifuged at 4000 rpm to obtain supernatant as ascites of monoclonal antibody.

2. Monoclonal antibody purification: ascites were centrifuged for 15 min (4000 rpm, room temperature), supernatant was taken, and saturated ammonium sulfate was slowly added drop by drop under stirring at 4° C. add to semi-saturation; stirring was continued for 30 min, centrifugation was performed for 30 min (13000 rpm, 4° C.), and supernatant was discarded; precipitate was dissolved in an appropriate amount of PBS(0.01 M, pH7.4), and slowly added with saturated ammonium sulfate drop by drop to 33% under stirring at 4° C.; continue stirring was continued for 30 min, centrifugation was performed for 30 min (13000 rpm, 4° C.), and supernatant was discarded. Precipitate was dissolved in an appropriate amount of PBS (0.01 M, pH 7.4), dialyzed at 4° C. overnight, antibody content was determined, and frozen at -20° C. for reserve. After ammonium sulfate precipitation, Protein G column was used for purification. New column was first passed with 5 mL ultrapure water, and then balanced with 5 mL 0.4 M PB buffer (pH 7.0) to purify the column; antibody was passed through the column with requirement of slow passing through the column in process, in order to better bind antibody protein on binding site; the column was subsequently equilibrated with 10 mL 0.4 M PB buffer (pH 7.0); the antibody at the binding site was eluted with 5 ml of 0.1 M glycine-hydrochloric acid buffer (pH 2.7) and glycine was neutralized by adding 1 M Tris-HCl (pH 8.0) to maintain a neutral pH suitable for antibody preservation.

### Antibody Screening

1. Titer detection Titer of purified antibody was detected by indirect ELISA method, and data were shown in Table 2:

TABLE 2

| | | | | | | | | | |
|---|---|---|---|---|---|---|---|---|---|
| Antibody number | 100 | 1K | 4K | 16K | 64K | 256K | 1024K | Negative | Titer |
| FL484-1 | 2.926 | 2.909 | 2.866 | 2.823 | 2.760 | 2.552 | 2.232 | 0.169 | > 1:1024k |
| FL484-2 | 2.876 | 2.871 | 2.835 | 2.707 | 2.647 | 2.516 | 1.969 | 0.208 | > 1:1024k |
| FL484-3 | 2.847 | 2.833 | 2.797 | 2.739 | 2.659 | 2.453 | 1.938 | 0.140 | > 1:1024k |
| FL484-4 | 2.836 | 2.822 | 2.656 | 2.556 | 2.192 | 1.436 | 0.931 | 0.230 | > 1:1024k |
| FL484-5 | 2.882 | 2.803 | 2.789 | 2.778 | 2.718 | 2.220 | 1.951 | 0.237 | > 1:1024k |
| FL484-6 | 2.802 | 2.760 | 2.759 | 2.708 | 2.694 | 2.172 | 2.131 | 0.240 | > 1:1024k |
| FL484-7 | 2.960 | 2.936 | 2.936 | 2.927 | 2.881 | 2.699 | 2.462 | 0.240 | > 1:1024k |
| FL484-8 | 2.790 | 2.762 | 2.737 | 2.692 | 2.654 | 2.291 | 2.538 | 0.196 | > 1:1024k |
| FL484-9 | 2.484 | 2.460 | 2.458 | 2.439 | 2.438 | 2.362 | 2.176 | 0.171 | > 1:1024k |
| FL484-10 | 2.737 | 2.728 | 2.726 | 2.696 | 2.635 | 2.344 | 1.613 | 0.158 | > 1:1024k |
| FL484-11 | 2.544 | 2.509 | 2.503 | 2.484 | 2.460 | 2.292 | 1.835 | 0.052 | > 1:1024k |
| FL484-12 | 2.837 | 2.755 | 2.742 | 2.727 | 2.705 | 2.679 | 2.647 | 0.157 | > 1:1024k |
| FL484-13 | 2.660 | 2.526 | 2.548 | 2.390 | 2.236 | 2.180 | 1.718 | 0.155 | > 1:1024k |
| FL484-14 | 2.593 | 2.381 | 2.361 | 2.270 | 2.133 | 1.733 | 1.083 | 0.190 | > 1:1024k |
| FL484-15 | 2.730 | 2.546 | 2.511 | 2.444 | 2.250 | 1.943 | 1.469 | 0.187 | > 1:1024k |
| FL484-16 | 2.597 | 2.558 | 2.516 | 2.355 | 2.317 | 2.027 | 1.577 | 0.209 | > 1:1024k |
| FL484-17 | 2.763 | 2.745 | 2.532 | 2.529 | 2.234 | 1.693 | 1.087 | 0.186 | > 1:1024k |
| FL484-18 | 2.747 | 2.660 | 2.513 | 2.426 | 2.240 | 1.712 | 1.577 | 0.179 | > 1:1024k |
| FL484-19 | 2.782 | 2.740 | 2.648 | 2.620 | 2.528 | 2.401 | 2.232 | 0.164 | > 1:1024k |
| FL484-20 | 2.798 | 2.794 | 2.729 | 2.560 | 2.394 | 2.187 | 2.076 | 0.236 | > 1:1024k |
| FL484-21 | 2.571 | 2.478 | 2.431 | 2.348 | 2.288 | 2.184 | 2.001 | 0.180 | > 1:1024k |
| FL484-22 | 2.925 | 2.936 | 2.843 | 2.760 | 2.522 | 2.043 | 1.543 | 0.163 | > 1:1024k |
| FL484-23 | 2.655 | 2.455 | 2.251 | 2.212 | 2.193 | 1.921 | 1.286 | 0.232 | > 1:1024k |
| FL484-24 | 2.767 | 2.723 | 2.522 | 2.452 | 2.232 | 1.840 | 1.221 | 0.203 | > 1:1024k |
| FL484-25 | 2.421 | 2.412 | 2.306 | 2.328 | 2.115 | 1.764 | 1.411 | 0.234 | > 1:1024k |
| FL484-26 | 2.484 | 2.432 | 2.308 | 2.303 | 2.050 | 1.550 | 1.042 | 0.161 | > 1:1024k |
| FL484-27 | 2.469 | 2.451 | 2.287 | 2.289 | 2.120 | 1.682 | 1.167 | 0.197 | > 1:1024k |
| FL484-28 | 2.524 | 2.487 | 2.351 | 2.363 | 2.190 | 1.824 | 1.720 | 0.229 | > 1:1024k |
| FL484-29 | 2.591 | 2.480 | 2.274 | 1.825 | 1.137 | 1.189 | 0.969 | 0.215 | > 1:1024k |
| FL484-30 | 2.544 | 2.533 | 2.449 | 2.382 | 2.414 | 2.078 | 1.812 | 0.265 | > 1:1024k |
| FL484-31 | 2.645 | 2.570 | 2.484 | 2.359 | 2.355 | 2.105 | 1.936 | 0.172 | > 1:1024k |
| FL484-32 | 2.773 | 2.703 | 2.664 | 2.451 | 2.407 | 2.151 | 2.110 | 0.194 | > 1:1024k |
| FL484-33 | 2.469 | 2.385 | 2.219 | 2.108 | 1.940 | 1.427 | 1.863 | 0.221 | > 1:1024k |
| FL484-34 | 2.716 | 2.541 | 2.453 | 2.268 | 2.236 | 1.662 | 1.033 | 0.193 | > 1:1024k |
| FL484-35 | 2.492 | 2.480 | 2.456 | 2.363 | 2.216 | 2.133 | 1.851 | 0.190 | > 1:1024k |

2. Subtype detection Mouse antibody subtype detection kit was used for subtype detection of antibodies, and results were shown in Table 3 and Table 4:

TABLE 3

| Antibody number | IgA | IgM | IgG1 | IgG2a | IgG2b | IgG3 | κ | λ |
|---|---|---|---|---|---|---|---|---|
| FL484-0 1 | 0.185 | 0.234 | 1.76 | 0.189 | 0.15 | 0.149 | 1.355 | 0.145 |
| FL484-02 | 0.161 | 0.209 | 1.903 | 0.168 | 0.195 | 0.134 | 1.477 | 0.127 |
| FL484-03 | 0.189 | 0.200 | 1.85 | 0.218 | 0.137 | 0.129 | 1.371 | 0.129 |
| FL484-04 | 0.161 | 0.223 | 1.949 | 0.148 | 0.135 | 0.129 | 1.413 | 0.124 |
| FL484-05 | 0.183 | 0.192 | 1.901 | 0.146 | 0.126 | 0.117 | 1.372 | 0.146 |
| FL484-06 | 0.181 | 0.227 | 2.12 | 0.166 | 0.129 | 0.134 | 1.537 | 0.134 |
| FL484-07 | 0.152 | 0.176 | 1.959 | 0.165 | 0.116 | 0.103 | 1.386 | 0.124 |
| FL484-08 | 0.229 | 0.201 | 2.000 | 0.177 | 0.105 | 0.121 | 1.397 | 0.124 |
| FL484-09 | 0.179 | 0.195 | 1.953 | 0.161 | 0.137 | 0.102 | 1.341 | 0.133 |
| FL484-10 | 0.244 | 0.244 | 2.002 | 0.167 | 0.096 | 0.123 | 1.451 | 0.137 |
| FL484-11 | 0.197 | 0.271 | 2.001 | 0.177 | 0.112 | 0.125 | 1.387 | 0.157 |
| FL484-12 | 0.262 | 0.271 | 2.024 | 0.185 | 0.110 | 0.154 | 1.489 | 0.158 |
| FL484-13 | 0.073 | 0.058 | 1.555 | 0.057 | 0.058 | 0.054 | 1.328 | 0.075 |
| FL484-14 | 0.059 | 0.054 | 1.816 | 0.053 | 0.052 | 0.050 | 1.375 | 0.063 |
| FL484-15 | 0.07 | 0.051 | 1.831 | 0.054 | 0.053 | 0.047 | 1.312 | 0.052 |
| FL484-16 | 0.091 | 0.055 | 1.639 | 0.055 | 0.053 | 0.050 | 1.312 | 0.056 |
| FL484-17 | 0.065 | 0.058 | 1.742 | 0.055 | 0.050 | 0.050 | 1.346 | 0.061 |
| FL484-18 | 0.079 | 0.058 | 1.726 | 0.055 | 0.053 | 0.049 | 1.311 | 0.057 |
| FL484-19 | 0.077 | 0.061 | 1.867 | 0.054 | 0.045 | 0.043 | 1.358 | 0.052 |
| FL484-20 | 0.087 | 0.059 | 1.784 | 0.053 | 0.047 | 0.045 | 1.310 | 0.050 |
| FL484-21 | 0.080 | 0.057 | 1.946 | 0.056 | 0.051 | 0.048 | 1.411 | 0.052 |
| FL484-22 | 0.077 | 0.059 | 1.954 | 0.054 | 0.048 | 0.047 | 1.322 | 0.058 |
| FL484-23 | 0.083 | 0.056 | 1.661 | 0.054 | 0.052 | 0.051 | 1.381 | 0.053 |
| FL484-24 | 0.08 | 0.056 | 1.875 | 0.058 | 0.049 | 0.051 | 1.337 | 0.056 |
| FL484-25 | 0.106 | 0.075 | 1.741 | 0.070 | 0.063 | 0.057 | 1.390 | 0.061 |
| FL484-26 | 0.08 | 0.064 | 1.597 | 0.058 | 0.055 | 0.052 | 1.338 | 0.064 |
| FL484-27 | 0.092 | 0.076 | 1.744 | 0.063 | 0.056 | 0.049 | 1.398 | 0.060 |
| FL484-28 | 0.176 | 0.125 | 2.134 | 0.091 | 0.067 | 0.074 | 1.125 | 0.095 |
| FL484-29 | 0.138 | 0.079 | 2.056 | 0.061 | 0.150 | 0.062 | 1.065 | 0.066 |
| FL484-30 | 0.223 | 0.206 | 1.580 | 0.183 | 0.177 | 0.195 | 1.084 | 0.163 |
| FL484-31 | 0.183 | 0.135 | 1.643 | 0.182 | 0.183 | 0.207 | 0.985 | 0.184 |
| FL484-32 | 0.135 | 0.155 | 0.077 | 0.088 | 1.631 | 0.107 | 1.08 | 0.109 |
| FL484-33 | 0.001 | 0.163 | 1.642 | 0.183 | 0.162 | 0.221 | 1.000 | 0.104 |
| FL484-34 | 0.228 | 0.153 | 1.628 | 0.201 | 0.176 | 0.186 | 0.982 | 0.185 |
| FL484-35 | 0.201 | 0.171 | 1.650 | 0.174 | 0.165 | 0.166 | 0.801 | 0.191 |

TABLE 4

| | | | | |
|---|---|---|---|---|
| Cell number | Clone number | Subtypes | Light chain or heavy chain | Ascites titer |
| FL484-0 1 | 1G3-1G8-1H1 | IgG1 | K | >1: 1024K |
| FL484-02 | 1E6-2D1-1H9 | IgG1 | K | >1: 1024K |
| FL484-03 | 1H11-2E7-2D5 | IgG1 | K | >1: 1024K |
| FL484-04 | 2B3-3E2-2H9 | IgG1 | K | >1: 1024K |
| FL484-05 | 2B4-4C4-3B9 | IgG1 | K | >1: 1024K |
| FL484-06 | 2A6-4G8-4B2 | IgG1 | K | >1: 1024K |
| FL484-07 | 2F10-5F7-4D8 | IgG1 | K | >1: 1024K |
| FL484-08 | 3C3-7H9-6A2 | IgG1 | K | >1: 1024K |
| FL484-09 | 3E5-8B12-6F7 | IgG1 | K | >1: 1024K |
| FL484-10 | 4D2-9B3-7D5 | IgG1 | K | >1: 1024K |
| FL484-11 | 7F8-18D9-15C4 | IgG1 | K | >1: 1024K |
| FL484-12 | 8A1-19G5-15A8 | IgG1 | K | >1: 1024K |
| FL484-13 | 8F9-21D4-17D3 | IgG1 | K | >1: 1024K |
| FL484-14 | 10E2-23A7-18E4 | IgG1 | K | >1: 1024K |
| FL484-15 | 7B4-17D1-14B5 | IgG1 | K | >1: 1024K |
| FL484-16 | 10C7-24F8-19H2 | IgG1 | K | >1: 1024K |
| FL484-17 | 10B11-25B8-19C7 | IgG1 | K | >1: 1024K |
| FL484-18 | 5H6-13D 10-10C8 | IgG1 | K | >1: 1024K |
| FL484-19 | 4D8-10E2-8G1 | IgG1 | K | >1: 1024K |
| FL484-20 | 4E9-10G7-8H8 | IgG1 | K | >1: 1024K |
| FL484-21 | 4A10-11A3-9A2 | IgG1 | K | >1: 1024K |
| FL484-22 | 5G3-12D9-9H8 | IgG1 | K | >1: 1024K |
| FL484-23 | 5D5-13H3-10G6 | IgG1 | K | >1: 1024K |
| FL484-24 | 5H7-14G3-11C1 | IgG1 | K | >1: 1024K |
| FL484-25 | 6D2-15B2-12G2 | IgG1 | K | >1: 1024K |
| FL484-26 | 7G3-16H9-13B7 | IgG1 | K | >1: 1024K |
| FL484-27 | 3F2-6F7-5G2 | IgG1 | K | >1: 1024K |
| FL484-28 | 7F6-18E1-14H8 | IgG1 | K | >1: 1024K |
| FL484-29 | 8D8-20B8-16E7 | IgG1 | K | >1: 1024K |
| FL484-30 | 9A7-21E11-17H7 | IgG1 | K | >1: 1024K |
| FL484-31 | 10D4-24E1-18D10 | IgG1 | K | >1: 1024K |
| FL484-32 | 4H4-9E10-7E8 | IgG2b | K | >1: 1024K |
| FL484-33 | 5E12-14H8-11E8 | IgG1 | K | >1: 1024K |
| FL484-34 | 6H7-16G1-13C3 | IgG1 | K | >1: 1024K |
| FL484-35 | 6C7-15F8-12F10 | IgG1 | K | >1: 1024K |

3. Antibody pairing The obtained antibody was screened by gold labeled method of double antibody sandwich, and the antibody pairs which could be paired were screened out.

Among them, FL484-03 and FL484-10 had the best pairing effect, so this pair of antibodies was selected as antibody for preparing subsequent colloidal gold rapid test strip, and this pair of antibodies was deposited.

4. Strip specificity detection Cross reaction of TMV, PVY, TSWV, ChiVMV and other common tobacco viruses was detected, and results showed that there was no cross reaction between the antibodies and other plant viruses. The result was shown in FIG. 2, from left to right, there were TMV, PVY, TSWV, ChiVMV, TZSV positive leaf samples determined by RT-PCR.

Experimental conclusion: The test strip provided by the invention has specificity for TZSV, and no specific reaction to other viruses like TMV, PVY, TSWV and ChiVMV

### Example 3. TZSV Hand-Held Colloidal Gold Rapid Test Strip Production

TZSV hand-held colloidal gold rapid test strip provided in this example (TZSV virus immunoassay test card) consists of a sample pad, gold labeled pad, nitrocellulose membrane, absorbent pad (absorption pad) and a backplane; sample pad, gold labeled pad, nitrocellulose membrane and absorbent pad are successively superimposed and pasted on the backplane, and each part is superimposed between each other by 2.5 mm; the gold labeled pad was coated with colloidal gold labeled anti-TZSV monoclonal antibody; nitrocellulosic membrane is provided with a test line and a quality control line (T line and C line), the test line is coated with anti-TZSV monoclonal antibody, the quality control line is coated with anti-mouse IgG secondary antibody.

Wherein, the anti-TZSV monoclonal antibody was secreted and produced by hybridoma cell lines with depository numbers of CCTCC No. C202160 and CCTCC No. C202196.

Concentration of anti-mouse IgG secondary antibody coated on the quality control line was 1 mg/ml. Size of the sample pad is 3 mm × 15 mm, size of the gold labeled pad is 3 mm×3 mm, size of the nitrocellulose membrane is 3 mm×28 mm, and size of the absorption pad is 3 mm×19 mm.

Space between the test line and the quality control line is 6-6.5 mm.

The backplane (bottom plate) is a PVC board.

Production method of the test card is as follows:
1. Preparation of nitrocellulose membrane containing test line T and quality control line C
   1) Preparation of coated murine monoclonal antibody
      TZSV murine monoclonal antibody was diluted to 1 mg/mL with PBS buffer solution of 10 mM and pH 7.4 to obtain TZSV murine monoclonal antibody solution, which was used as coated murine monoclonal antibody for test line T.
      Sheep anti-mouse IgG secondary antibody solution was diluted to 1 mg/ mL in PBS buffer solution with concentration of 10 mM and pH of 7.4, which was used as coated mouse monoclonal antibody for quality control line C.
   2) Coat
      Nitrate cellulose membrane (NC film) of PALL170 was selected, and TZSV murine monoclonal antibody solution with a concentration of 1.0 mg/mL was drawn T line at 1.0 µL/cm with Dispenser and Sprayer, which was used as the test line; sheep anti-mouse IgG secondary antibody solution with a concentration of 1 mg/mL was drawn C line with 1.0 µL/cm as the quality control line; they were dried at 37° C. for 24 hours and stand-by; the nitrocellulose membrane coated with murine monoclonal antibody was obtained.
2. Preparation of gold labeled pad
   1) Colloidal gold preparation
      a) Preparation: 500 mL beaker, 20 mL small beaker, rotor, brown bottle, glass rod, etc. were washed out, put into acid cylinder (potassium bichromate: concentrated sulfuric acid: ultra-pure water = 120 g:200 ml: 1000 ml) and soaked for 24 hours. They were taken out, rinsed 3-4 times with tap water, then rinsed 3-4 times with ultrapure water, and put in oven to dry at 37° C. for reserve.
      b) preparation of gold burning solution A: 1 g chloroauric acid powder (purchased from Sigma) was weighed with a plastic weighing spoon in a brown bottle, fully dissolved by adding 99 mL ultra-pure water, and stored at 4° C. with avoiding light.
      c) preparation of gold burning solution B: 1 g trisodium citrate (purchased from Sigma) was weighed, dissolved in 99 mL ultra-pure water and mixed well.
      d) Preparation of colloidal gold: 99 ml ultrapure water was measured out in beaker, 1 ml gold burning solution A was added; mixture were stirred and mixed well on constant temperature magnetic stirrer; mixture was heated to boiling, 2 ml newly prepared burning gold solution B was added quickly, stirred and heated subsequently; solution gradually became a blue-black, then purple black; when reheating solution appeared red, and became transparent orange red by continuously boiling; solution was continuously boiled for 10 min, cooled naturally to room temperature, and added ultrapure water to 100 ml. Solution was poured into a brown bottle and stored at 4° C. with avoiding light; the colloidal gold solution was obtained (particle size of colloidal gold was 40 nm, and concentration is one part in ten thousand).
   2) Murine monoclonal antibody labeling
      e) Labeling of murine monoclonal antibody: 1.5 ml of colloidal gold solution prepared by above step 1) was taken, adjusted pH value with 0.1 M K₂CO₃, added 20 µg TZSV monoclonal murine monoclonal antibody, mixed well, and reacted at room temperature for 40 min. Reaction was terminated by adding 10% BSA and placed for 30 min.
      f) Purification of labeled murine monoclonal antibody: products obtained by above step e) was centrifuged at low speed (1500 r/min), precipitation formed by condensed gold colloides was discarded, and supernatant was collected; such supernatant was centrifuged at high speed (8500 r/min) for 30 min, with supernatant carefully removed, and precipitate was collected. Precipitate was redissolved in 0.1 M PBS (pH 7.4) containing 1% (mass percentage) BSA and stored at 4° C. The labeled colloidal gold TZSV murine monoclonal antibody solution was obtained with concentration of 0.4 mg/ml.
   3) Spray gold
      The labeled colloidal gold TZSV mouse monoclonal antibody solution with concentration of 0.4 mg/ mL prepared by above step 2) was sprayed on the pretreated gold labeled pad with 1.0 µL/cm, dried and ready for use, and the gold label pad fixed with colloidal gold labeled specific monoclonal mouse monoclonal antibody was obtained.
3. Assembly and cutting
   The sample pad, gold labeled pad fixed with colloidal gold labeled anti-TZSV monoclonal antibody, nitrocellulose membrane and absorbent pad were pasted onto the backplane in turn, and cut into 3 mm wide with a strip cutter to obtain the TZSV colloidal gold rapid test strip.

### Example 4. Evaluation on Effect of TZSV Virus Immunoassay Test Card in Detecting Virions

1. 200 µl blank sample solution (pH 7.4, 0.2 M/L PBS solution) was added into the sample cup, and the preparation method was as follows: 8 g sodium chloride, 3.35 g disodium hydrogen phosphate dodecahydrate, 0.2 g potassium dihydrogen phosphate and 0.2 g potassium chloride was dissolved by double steamed water to a constant volume of 1 L. The test strip was inserted into the sample cup rapidly, and the color development results were observed in the result observation area after 8 minutes of reaction at room temperature. The results show that T line did not produce color in the observation area, while C line did.

2. 100 µl of 0.1 µg/ml TZSV virus standard (prepared with above PBS solution) was added into the sample cup, and the same procedure as the blank sample was performed.

The results show that C line and T line produce color simultaneously in the observation area. Experimental conclusion: The TZSV virus detection reagent of the invention can quickly detect TZSV virus, and has high sensitivity and convenient operation.

### Example 5. Evaluation on Effect of TZSV Virus Immunoassay Card on Real Leaf Samples

1. 200 µl of negative leaf sample extract was added into the sample cup and performed according to the same procedure as the standard (same as step 1 of example 4). The results show that only the control line C produced color in the observation area, while the test line T did not.

2. 200 µl positive leaf sample extract was added into the sample cup, and performed according to the same procedure as negative sample.

The results show that both the control line C and the test line T produce color in the observation area. Experimental conclusion: The TZSV virus hand-held colloidal gold speed test strip of the invention has good anti-interference effect on leaf and other samples, and can be widely used in rapid detection of TZSV, with high sensitivity and convenient operation.

### Example 6. Preparation and Performance Verification of TZSV Test Strip

### 6.1 Prokaryotic Expression of N Protein in Yunnan Province TZSV Lines

This example performed theoretical evaluation on hydrophilicity or hydrophobicity, signal peptide, transmembrane domain, basic structure of N protein in TZSV Yunnan strain, plasmid construction program was designed according to results of the evaluation with method of full gene synthesis to construct plasmid, clone into expression vector, further transform into a competent escherichia coli cells, culture, induced express, collect bacteria, purify protein. Finally, 5 mg recombinant TZSV-N protein with purity >90% was obtained and verified by SDS-PAGE.

Gene sequence with optimized codon was shown in SEQ ID NO.2, with 6 bases at 5 ‘end and 6 bases at 3’ end as restriction sites.

Amino acid sequence of prokaryotic expressed TZSV-N Protein was shown in SEQ ID NO.1. Protein Length=282 AA, MW= 31 KDa (34 KDa with label), Predicted pI= 8.6.

Protein expression and verification

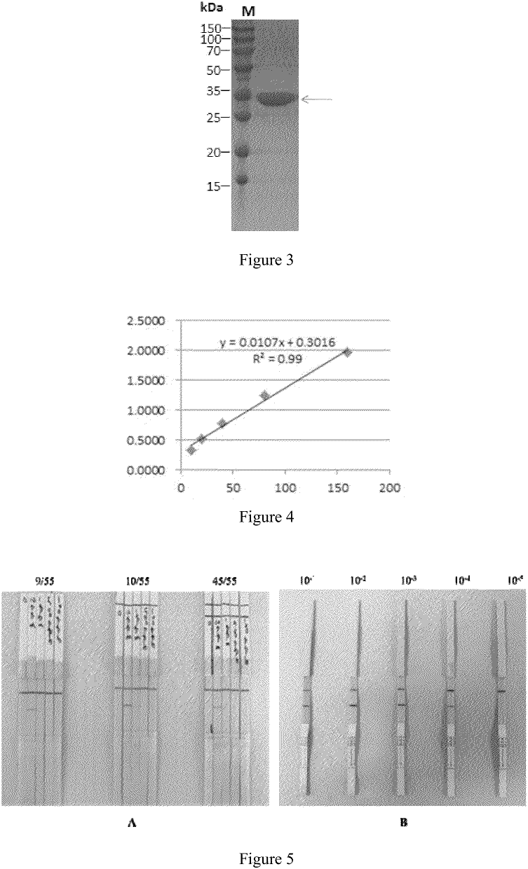

After purification, fusion protein can be expressed in a large amount in supernatant, and it has been proved that the maximum amount of the fusion protein can be purified by elution of imidazole at a concentration of 200-500 mM. It’s preliminarily to determine that the fusion protein was purified successfully when results of SDS-PAGE showed that there were obvious bands in the corresponding position of theoretical molecular weight ±5 kDa. Electrophoresis results of pET28a-TZSV-N protein purification are shown in FIG. 3.

TZSV N protein was successfully expressed and purified by pET28a vector. After purification, 10 ml of target protein was obtained at a concentration of 0.5 mg/ml, whose total weight was 5 mg, and was packed according to 1 ml/tube.

Protein storage buffer: 20 mM Tris-Cl, 50 mM NaCl, 1 mM EDTA, pH=8.0.

### 6.2 Preparation of TZSV Mab

Four 6-week-old female BALB/ C mice were immunized for three times by subcutaneous multipoint injection. Antigen dosage of each mouse was 25 µg, the first immunization was emulsified with same volume of Freund’s complete adjuvant, the second immunization was emulsified with same volume of Freund’s incomplete adjuvant, and the third immunization was mixed with same volume of normal saline and injected intraperitoneally. Specific immunization procedures are shown in Table 5.

TABLE 5

preparation of immunization protocol of monoclonal antibodies preparation

| Number of immune | Date of immune | Interval | Adjuvant type | Site and method of immunity |
|---|---|---|---|---|
| First time | 2020/4/25 | 0 | Freund’s complete adjuvant | Subcutaneous multipoint injection |
| Second time | 2021/5/8 | 13 | Freund’s incomplete adjuvant | Subcutaneous multipoint injection |
| Third time | 2021/5/28 | 20 | Physiological saline | Intraperitoneal injection |

Twenty days after the second immunization, blood was collected from tail vein and antibody titers were measured by an indirect method to determine whether antibodies against antigen had been generated. Titers of four mice were compared, and the mouse with higher antibody titer was finally selected for cell fusion (mouse number: M210045). Three days before fusion, booster immunization was performed directly with antigen and with same dose as before.

A total of 37 positive monoclonal hybridoma cell lines were screened and established. The hybridoma cells were collected, and mouse ascites were prepared and collected, the 1000-fold dilution of TZSV positive samples collected in field was used as sample to be tested, and antibody titer was determined by indirect ELISA method. The titer can be as high as 1:1024000. Results of monoclonal antibody titer detection are shown in Table 6.

TABLE 6

TZSV N protein mouse monoclonal antibody assay (partial)

| | | | | | | | | | |
|---|---|---|---|---|---|---|---|---|---|
| Cell number | Detection original | Coated concentration | 100 | 16K | 64K | 256K | 1024K | Titer | Negative samples |
| FL484-9 | Positive tobacco | 0.74 | 1.82 | 1.24 | 1.15 | 0.98 | 0.62 | 1024K | 0.39 |
| FL484-11 | Positive tobacco | 0.74 | 1.18 | 0.79 | 0.64 | 0.36 | 0.25 | 64K | 0.29 |
| FL484-19 | Positive tobacco | 0.74 | 1.32 | 0.76 | 0.56 | 0.37 | 0.26 | 64K | 0.29 |
| FL484-21 | Positive tobacco | 0.74 | 1.20 | 0.78 | 0.58 | 0.36 | 0.26 | 64K | 0.20 |
| FL484-34 | Positive tobacco | 0.74 | 1.80 | 0.78 | 0.53 | 0.30 | 0.23 | 64K | 0.37 |
| FL484-35 | Positive tobacco | 0.74 | 1.34 | 0.96 | 0.75 | 0.48 | | 64K | 0.24 |

### 6.3 TZSV ELISA Kit Development

Antibody with good performance in preliminary experiment was selected to be paired with polyclonal antibody, the results in Table 7 showed that the monoclonal antibody with No. 3#, 9#, 10, 21, 37 could detect recombinant protein with a sensitivity of more than 10 PPB when they were paired with polyclonal antibody, and all of them could detect TZSV tobacco leaf samples that were pathogenetic in field; 9# monoclonal antibody was selected for subsequent enzyme-labeled product optimization.

TABLE 7

Detection on effect of TZSV monoclonal antibody

| Mab number | Recombinant protein | | | | Positive tobacco leaf sample 1 | | Positive tobacco leaf sample 2 | |
|---|---|---|---|---|---|---|---|---|
| | 1 ppm | 100 ppb | 10 ppb | 0 | 10x | 100x | 10x | 100x |
| 3# | 3.7328 | 1.385 | 0.1943 | 0.0741 | 3.5855 | 3.4265 | 1.4824 | 1.3598 |
| 9# | 3.7364 | 1.5564 | 0.3963 | 0.2371 | 3.5519 | 3.6975 | 3.4816 | 3.5113 |
| 10# | 3.7521 | 2.9797 | 0.66 | 0.059 | 2.7301 | 2.4701 | 1.8265 | 1.483 |
| 21# | 3.8608 | 2.3165 | 0.3963 | 0.1149 | 3.5117 | 3.5395 | 2.6189 | 2.4474 |
| 37# | 3.9104 | 2.6782 | 0.5833 | 0.1469 | 3.4838 | 3.4129 | 2.802 | 2.5955 |

9# monoclonal antibody was selected as the coated antibody, and standard product with gradient concentration was prepared through condition optimization. Linear range of 10-160 ppb was selected as detection range (Table 8), and standard curve was obtained through linear regression (FIG. 4).

TABLE 8

Optimization conditions of 9# monoclonal antibody

| | | ODI | OD2 | Average OD |
|---|---|---|---|---|
| Recombinant protein ppb | 320 | 2.8612 | 2.8861 | 2.8737 |
| | 160 | 2.0368 | 1.8921 | 1.9645 |
| | 80 | 12554 | 1.2249 | 1.2402 |
| | 40 | 0.8287 | 0.7174 | 0.7731 |
| | 20 | 0.5353 | 0.4904 | 0.5129 |
| | 10 | 345 | 0.3174 | 0.3312 |
| | 5 | 0.2531 | 0.2454 | 0.2493 |
| | 0 | 0.1956 | 0.1678 | 0.1817 |
| | Sample100 x | 3.5307 | 34329 | 3.4818 |
| | Sample IK x | 0.98 | 0.9962 | 0.9881 |
| | Sample10K x | 0.253 | 0.2478 | 0.2504 |
| | Negative samples | 0.1448 | 0.1467 | 0.1458 |

### 6.4 Preparation of TZSV Test Strip

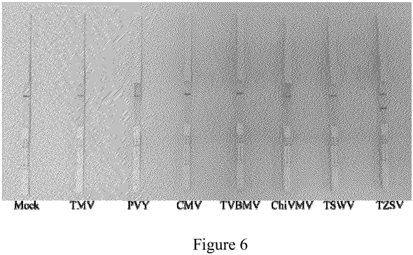

Firstly, sensitivity of test strip was tested. As shown in FIG. 5, from left to right, the three groups are No. 9 monoclonal antibody labeled gold/No. 55 monoclonal antibody spray film, No. 10 monoclonal antibody labeled gold/No. 55 monoclonal antibody spray film, and No. 45 monoclonal antibody labeled gold/No. 55 monoclonal antibody spray film. From left to right in each group there were 0, 10 ppm, 1 ppm, 500 ppb, and 100 ppb of recombinant proteins as antigens. The test strip of monoclonal antibody No. 10 standard gold/monoclonal antibody No. 55 spray film can reach result that line appears indistinctly at 500 ppb (FIG. 5A). TZSV positive tobacco leaf samples pathogenetic in field were used for sensitivity detection. Supernatant after grinding 0.1 g fresh pathogenetic leaves and adding 1 mL PBS to mix and centrifuge was used as 10⁻¹ times dilution of virus diluent, and 10⁻², 10⁻³, 10⁻⁴, and 10⁻⁵ times dilution of virus diluent were prepared successively. 150 µL dilution of each was used to test sensitivity of the test strip. Results showed that the test strip of No. 10 mab standard gold/No. 55 mab spray film had the best sensitivity, and the T-line was still clearly visible when virus diluent was diluted 10⁻⁴ times, while line appears indistinctly when virus diluent was diluted 10⁻⁵ times (FIG. 5B).

TMV, PVY, CMV, TVBMV, ChiVMV, TSWV and TZSV infected leaves which were identified as positive by RT-PCR were used as test samples to detect specificity of TZSV test strip. As shown in FIG. 6, only TZSV samples showed positive reaction, while other virus-positive samples showed negative results, indicating that TZSV test strip had good specificity.

Evaluation on stability of TZSV strip: Test strip was placed in an environment of 4° C., room temperature and 37° C. for 6 months, during this period, test strip was taken out to test 100-fold dilution of negative control and TZSV positive sample every other month, and results are shown in Table 9, during the stability evaluation period of 6 months, the test strip maintained good stability at three temperatures, and no false positives or false negatives occurred.

TABLE 9

TZSV test strip stability test

| treatment | | Time/Month | | | | | | |
|---|---|---|---|---|---|---|---|---|
| | | 0 | 1 | 2 | 3 | 4 | 5 | 6 |
| Positive | 4 ° | + | + | + | + | + | + | + |
| | Room temperature | + | + | + | + | + | + | + |
| | 37 ° | + | + | + | + | + | + | + |
| Negative | 4 ° | — | — | — | — | — | — | — |
| | Room temperature | — | — | — | — | — | — | — |
| | 37 ° | — | — | — | — | — | — | — |

1. Hybridoma cell lines whose depository numbers are CCTCC No. C202160 and CCTCC No. C202196.

2. Use of the hybridoma cell lines of claim 1 in preparing anti-tomato zonate spot virus monoclonal antibody.

3. Anti-tomato zonate spot virus monoclonal antibody generated by hybridoma cell lines whose depository numbers are CCTCC No. C202160 and CCTCC No. C202196.

4. Use of the monoclonal antibody of claim 3 in preparing tomato zonate spot virus detection reagent.

5. Gene with nucleic acid sequence as shown in SEQ ID NO:2.

6. Use of the gene of claim 5 in preparing anti-tomato zonate spot virus monoclonal antibody.

7. A reagent for detecting tomato zonate spot virus, characterized in that, comprising monoclonal antibody generated by the hybridoma cell lines of claim 1.

8. The reagent for detecting tomato zonate spot virus of claim 7 , is a test strip consisting of a sample pad, a gold labeled pad, a nitrocellulose membrane, an absorbent pad and a backplane; the sample pad, gold labeled pad, nitrocellulose membrane and absorbent pad are successively superimposed and pasted on the backplane, and each part is superimposed between each other by 2-3 mm; the gold labeled pad was coated with colloidal gold labeled anti-tomato zonate spot virus monoclonal antibody; nitrocellulose membrane is provided with a test line and a quality control line, the test line is coated with anti-tomato zonate spot virus monoclonal antibody, the quality control line is coated with anti-mouse IgG secondary antibody.

9. The reagent for detecting tomato zonate spot virus of claim 7, wherein the sample pad is glass cellulose membrane.

10. The reagent for detecting tomato zonate spot virus of claim 7, wherein concentration of colloidal gold labeled anti-tomato zonate spot virus monoclonal antibody coated on the gold labeled pad is 0.3-0.5 mg/ml.

11. The reagent for detecting tomato zonate spot virus of claim 7, wherein concentration of anti-tomato zonate spot virus monoclonal antibody coated by the test line is 0.5-1.5 mg/ml.

12. The reagents for detecting tomato zonate spot virus of claim 7, wherein the backplane is a PVC plate.


SEQUENCE LISTING (2 sequences)

SEQ ID NO: 1 - protein, 282 aa, unidentified
GSMSNVRSLTQQKVQELLAGGKADVEIDTDDQTQGFSFASFYEENKAKADFTYNTGINILKCRKQVFAACKNGKYEFCGHKIVASSADVSATDWTFKRTEAFIRTRLISMAEHATDETTKKQMYIKAMELPLVAAYGLNVPVDFNSSAIRLMLCIGGPLPLLSSVPGLAPVCFPLAYFQNVKKEQLGIKNFSTYEQICKVAKVLSAASVEFTEKTQELFTSTVKLLGESNPGTAGAISLHKYNDQLKQMETAFKSKLNVDDFGQNSKQAPKKKSSNDLSFLE

SEQ ID NO: 2 - DNA, 846 bp, unidentified
  source 1..846: mol_type = other DNA
ggatccatgagcaatgtgcgcagtctgacccagcagaaagtgcaggaactgctggcaggcggtaaagccgatgttgaaattgataccgatgatcagacccagggctttagttttgccagtttttatgaagaaaacaaggcaaaagcagattttacctataataccggtattaatatcctgaaatgccgcaaacaggtgtttgccgcctgcaaaaatggtaaatatgaattttgtggccacaaaattgtggccagtagcgcagatgtgagtgcaaccgattggacctttaaacgcaccgaagcctttattcgtacccgcctgattagtatggccgaacatgccaccgatgaaaccaccaaaaaacagatgtatattaaggcaatggaactgccgctggtggccgcatacggtctgaatgtgccggtggattttaatagtagtgcaattcgcctgatgctgtgtattggtggcccgctgccgctgctgagcagcgttcctggtctggccccggtgtgctttccgctggcatattttcagaatgtgaaaaaagaacagctgggcattaagaattttagtacctatgaacagatctgcaaagtggcaaaagtgctgagtgccgcaagcgttgagtttactgaaaaaacccaggaactgtttaccagtaccgttaaactgctgggcgaaagcaatccgggtacagcaggcgcaattagcctgcataaatataatgatcagctgaaacagatggaaaccgcattcaaaagcaaactgaatgtggatgattttggccagaatagcaaacaggcaccgaaaaagaaaagtagcaatgatctgagctttctcgag